(19) United States
(12) Patent Application Publication
(10) Pub. No.: US 2021/0370009 A1
(43) Pub. Date: Dec. 2, 2021

(54) HAND-HELD OXYGEN GENERATOR AND AIR PURIFIER

(71) Applicants: Richard Greenwood, Bolton (GB); Kim Myung-Suk, BUCHEON-SI (KR)

(72) Inventors: Richard Greenwood, Bolton (GB); Kim Myung-Suk, BUCHEON-SI (KR)

(21) Appl. No.: 16/882,775

(22) Filed: May 26, 2020

(51) Int. Cl. A61M 16/10; A61M 15/06; A61M 16/20; A61M 15/00; A63B 23/18; A61K 33/00; A24F 42/20; A24D 3/17; B01D 46/54; B01D 46/00; A24F 42/60; B01D 46/42

(52) U.S. Cl. CPC A61M 16/101; B01D 2279/40; A61M 16/208; A61M 16/107; A61M 15/0021; A63B 23/18; A61K 33/00; A24F 42/20; A24D 3/17; B01D 46/546; B01D 46/0023; B01D 46/0012; A24F 42/60; B01D 46/4272; A61M 2202/0208; B01D 2267/40; A61M 15/06

(57) ABSTRACT

Presented is a portable hand held e-cigarette shaped oxygen generator and air purifier. The device includes a suction body through which atmospheric air is forcibly introduced within a main body of the device during respiration, a solid oxygen filter adapted for holding solid oxygen, and a solid oxygen filter support having a dual cylindrical structure for housing and supporting the solid oxygen filter. The device further includes a solid oxygen filter support lid mountable over the solid oxygen filter support and is adapted for discharging device generated inhalable oxygen from the main body to a micro filter, a casing embodying the micro filter and operationally connected to a mouthpiece having an extension for mounting a straw. The atmospheric air introduced in the device reacts with the solid oxygen to generate inhalable oxygen after passing the atmospheric air through the dual cylindrical structure of the solid oxygen filter support.

### FIELD OF THE INVENTION

The present disclosure relates to oxygen generators and air purifiers, and more particularly, to a portable, light weight hand held oxygen-generating and air purifying device in which inhalable oxygen is generated by reacting solid oxygen with atmospheric air.

### BACKGROUND

Oxygen is the third most abundant element in the universe and makes up over 20% of the earth's atmosphere. In the human body, oxygen is the single most abundant element, making up 65% of body mass. We're breathing oxygen in the atmosphere nearly every second and it's a very important for human life for various reasons, such as for stress relief, increased memory and concentration, improved work performance, relaxation after exercise, and improved health of pregnant women and so on. Also, in order to breathe more oxygen, modern people's follow different forms of breathing such as yoga breathing, short-circulation breathing, double breathing, etc., which increase the diaphragm contraction and expansion.

The lack of oxygen concentration in air can lead to several health issues, sign/symptoms such as shown in Table 1 below:

TABLE 1

| Oxygen | |
|---|---|
| Content | |
| of Air (%) | Signs and Symptoms of persons at rest |
| 15-19.5 | Decreased ability to work strenuously. May impair |
| | coordination and may induce symptoms in persons with |
| | coronary, pulmonary, or circulatory problems. |
| 12-15 | Respiration deeper, increased pulse rate, impaired |
| | coordination, perception and judgement |
| 10-12 | Further increase in rate and depth of respiration, further |
| | increase in pulse rate, performance failure, giddiness, |
| | poor judgement and blue lips |
| 8-10 | Mental failure, nausea, vomiting, fainting, unconsciousness, |
| | ashen face, blue lips |
| 6-8 | 8 minutes, may be fatal in 50-100% of exposure; 6 minutes, |
| | may be fatal in 25-50% of exposure; 4-5 minutes, recovery |
| | with treatment |
| 4-6 | Coma in 40 seconds, convulsions, respiration ceases, death |

Earth's oxygen concentration is gradually decreasing due to the urbanization activities. Forests that produce most oxygen in the atmosphere are set to fire or getting destroyed due to urbanization. Several studies and research in the past has witnessed increased number of death tolls due to decrease in the concentration of oxygen in the atmosphere in the past few years.

Further, statistics from the Ministry of Health and Welfare 2017 show that about 22.3% of adults (19 years and above) smoke and many smokers habitually smoke while knowing that smoking is harmful to their health. Many smokers use e-cigarettes despite the government's strong anti-smoking policy. We are well aware about several cases where the users face injuries caused by explosion of e-cigarette devices in the United States and other countries. When a person breathes or smokes, he/she uses his/her lung and diaphragm to breathe like a vacuum pump.

Although solutions exist, such as Pressure Swing Adsorption (PSA) method/technology that may be cost-efficient and uses electricity for producing a high purity oxygen or nitrogen by compressing or pressurizing Zeolite pellets. PSA method has its own disadvantages of high purity and high noise. Further, the implementation of PSA method to extract oxygen or nitrogen requires larger setup involving two pressurized cylinders with molecular sieves to ensure continuous production, for separation of either oxygen or nitrogen from other gases, compressors, dryers, filter packages, air tanks and so on. Further, solutions exist, that uses solid oxygen compounds like calcium hydroxide and other oxides. Although, the method of generating oxygen has been used widely wherein oxygen is generated by reacting air with a compound composed of calcium, potassium peroxide, calcium peroxide, magnesium peroxide, potassium peroxide, barium peroxide and sodium peroxide, and carbon dioxide and moisture in the atmosphere. These available methods although do not require electricity or produce any noise, but has its own disadvantage that the amount of oxygen generated is insufficient due to a small contact area or reaction surface of carbon dioxide and moisture in the atmosphere

Further, several inventions/solutions are proposed in the past by several inventors for generating oxygen, for example, KR20140132994A discloses an oxygen generator using solid oxygen, which is capable of selectively generating oxygen generated naturally by contact between moisture in the atmosphere and solid oxygen. The oxygen generator according to this patent application includes an air suction means accommodated in the body of the oxygen generator for forcibly introducing outside air into the body through an air inlet, a water receiving unit for receiving water and generating moisture by the air introduced by the air suction unit, and an oxygen generating unit for receiving solid oxygen and communicating with the water receiving unit to generate oxygen by contacting the solid oxygen with moisture generated in the water receiving unit, and an oxygen generator using solid oxygen.

RU2697002C2 discloses a cigarette shaped portable oxygen generator for inhaling oxygen. According to the patent, the oxygen inhaling device uses ignition source to produce oxygen.

U.S. Pat. No. 7,513,251 discloses hand-held oxygen generating apparatus that includes a container containing potassium super oxide and valves that regulate oxygen and air flow.

Also, it is learnt that oxygen concentration in most of city centre areas is about 20.9%, and the oxygen concentration in the mountain is conceived to be about 22%. In other words, if we are able to increase the oxygen concentration by just only 1˜2%, we are able breathe comfortably like in the mountains.

In the light of aforementioned background information, inventors of the proposed invention herein proposes an e-cigarette shaped, portable, and light weight hand held oxygen-generating and air purifying device that can generate breathable oxygen by reacting solid oxygen compound with the atmospheric air, and also at the same time efficiently purify the generated air by removing harmful gases.

### SUMMARY

It is an objective of the present invention to provide e-cigarette shaped, portable, and light weight hand held oxygen-generating and air purifying device that uses solid oxygen compounds for generating inhalable/breathable oxygen generated by reacting the oxygen compound with the atmospheric air introduced within the device.

It is another objective of the present invention to provide an oxygen-generator comprising of dual filters (Micro filter and Solid Oxygen Filter).

Another objective of the present invention is to provide the solid oxygen carbon filter (embodied within the proposed oxygen generator) with a large reaction surface area and honeycomb-shaped perforated structure present within inner surface of the solid oxygen filter support.

Another objective of the present invention is to provide a refillable oxygen generator device that can be refilled with solid oxygen compound in the form of tablets and be used over and again.

Another objective of the invention is to provide an oxygen generator using which users can develop sound breathing/air inhalation pattern often required during air inhalation exercises. One can even use the proposed oxygen generator device to perform breathing exercises following some breathing exercise apps installed on their mobile devices.

In an embodiment of the present disclosure, there is provided a hand-held oxygen generator and air purifier. The portable hand held e-cigarette shaped oxygen-generating and air purifying device includes a suction body having a check valve a first filter, and a first casing through which atmospheric air is forcibly introduced within a main body of the device during respiration, the first filter and the check valve are mounted over the first casing, and wherein the first casing is operatively coupled to a distal end of the main body. The device further includes a solid oxygen filter adapted for holding solid oxygen therein, and a solid oxygen filter support having a dual cylindrical structure and configured for housing and supporting the solid oxygen filter, a solid oxygen filter support lid with a pair of hooks the solid oxygen filter support lid is mounted over the solid oxygen filter support, wherein the solid oxygen filter support lid further configured to discharge generated inhalable oxygen from the main body to a second filter.

According to the embodiment, the device further includes a second casing embodying the second filter and operationally connected to a mouthpiece and coupled to a proximal end of the main body, and wherein the mouthpiece having an extension for mounting a straw thereon. According to the embodiment, the atmospheric air introduced in the device by respiration reacts with the solid oxygen contained insider the solid oxygen filter to generate inhalable oxygen, and wherein the atmospheric air reacts with the solid oxygen after passing through the dual cylindrical structure of the solid oxygen filter support.

### BRIEF DESCRIPTION OF THE DRAWINGS

The following detailed description of the preferred embodiments of the present disclosure will be better understood when read in conjunction with the appended drawings. The present disclosure is illustrated by way of example, and not limited by the accompanying figures, in which like references indicate similar elements.

FIG. 1 shows a front perspective view of a portable hand held oxygen-generating and air purifying device, according to an embodiment;

FIG. 2 shows a back perspective view of the portable hand held oxygen-generating and air purifying device, according to an embodiment;

FIG. 3 shows a sectional view of the portable hand held oxygen-generating and air purifying device, according to an embodiment;

FIG. 4 shows an exploded view of the portable hand held oxygen-generating and air purifying device, according to an embodiment;

FIG. 5 shows an oxygen filter, a solid oxygen filter support having a dual cylindrical structure, and a solid oxygen filter support lid that fixedly attaches over the solid oxygen filter support;

FIG. 6 shows an inner cylinder (of the oxygen filter support) with a honeycomb perforated structure;

FIG. 7 illustrates section A shown in FIG. 4; and

FIG. 8 illustrates section B of FIG. 4.

### DETAILED DESCRIPTION

As used in the specification and claims, the singular forms “a”, “an” and “the” include plural references unless the context clearly dictates otherwise. For example, the term “an article” may include a plurality of articles unless the context clearly dictates otherwise. Those with ordinary skill in the art will appreciate that the elements in the figures are illustrated for simplicity and clarity and are not necessarily drawn to scale. For example, the dimensions of some of the elements in the figures may be exaggerated, relative to other elements, in order to improve the understanding of the present invention.

There may be additional components described in the foregoing application that are not depicted on one of the described drawings. In the event such a component is described, but not depicted in a drawing, the absence of such a drawing should not be considered as an omission of such design from the specification. Before describing the present invention in detail, it should be observed that the present invention utilizes a combination of components which constitutes a portable, light weight hand held oxygen-generating and air purifying device in which inhalable oxygen is generated by reacting solid oxygen with atmospheric air. Accordingly, the components and the method steps have been represented, showing only specific details that are pertinent for an understanding of the present invention so as not to obscure the disclosure with details that will be readily apparent to those with ordinary skill in the art having the benefit of the description herein.

As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

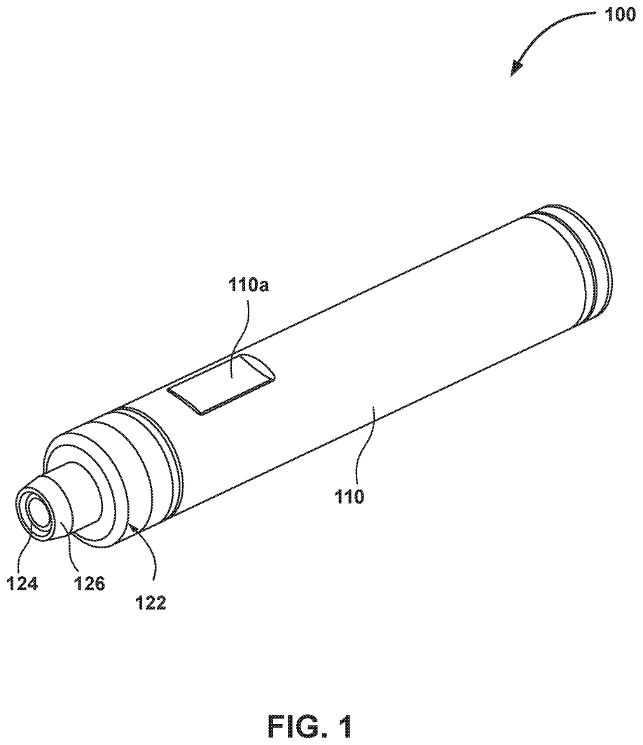

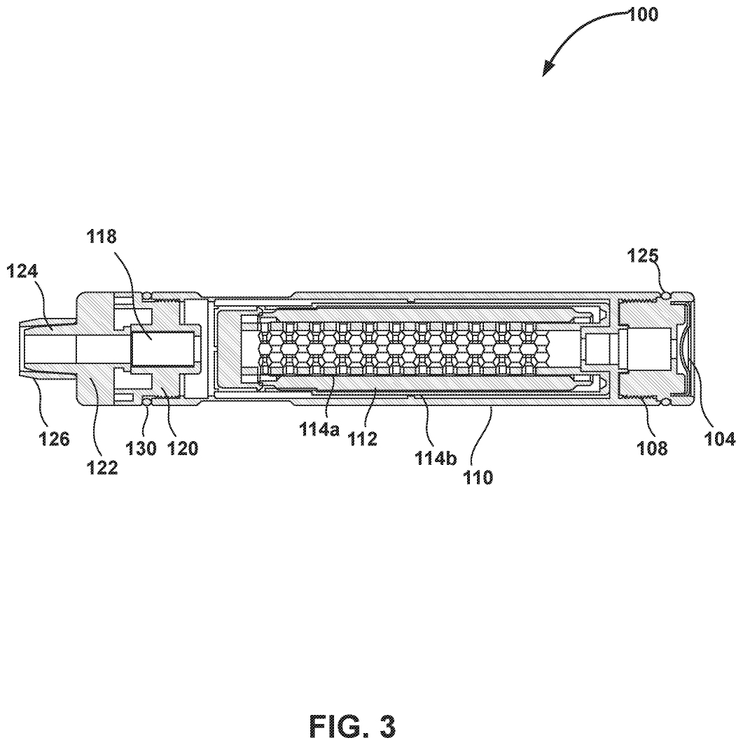

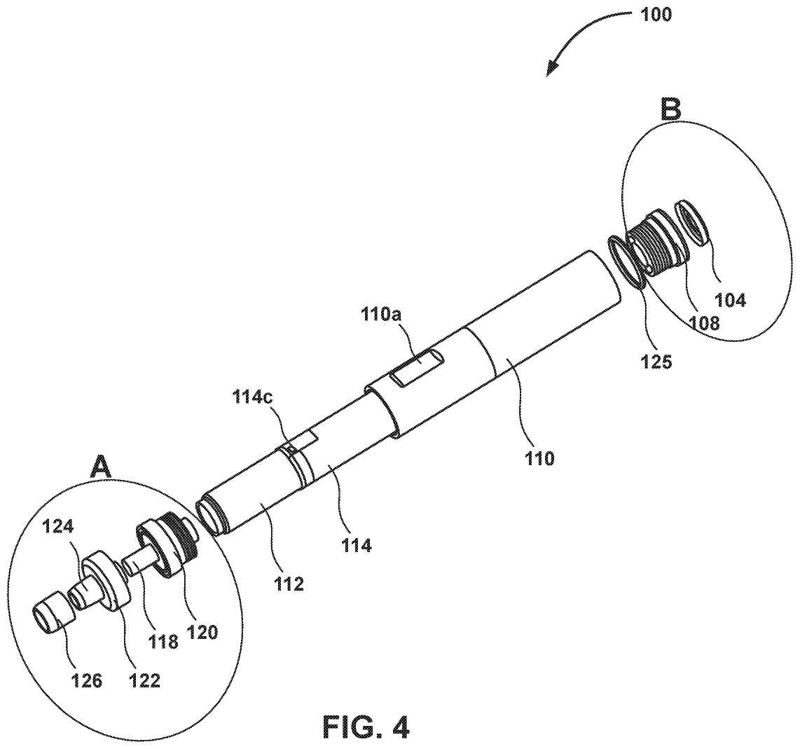

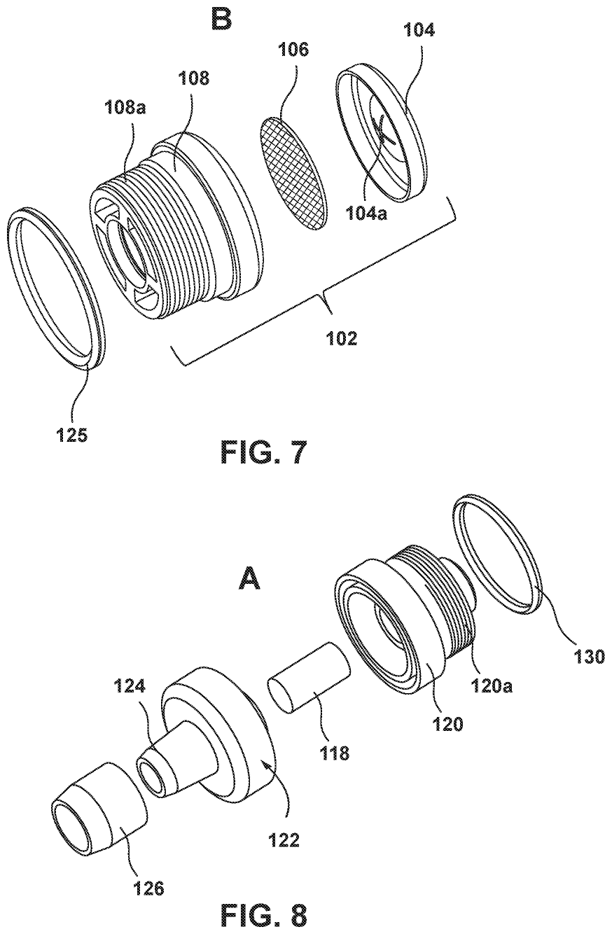

Referring now to accompanying figures, particularly FIGS. 1-8, there is provided a hand held oxygen generating and air purifying device 100. The proposed device 100 is shaped like an e-cigarette. The device 100 includes a suction body 102 (represented as Section ‘B’ in FIG. 4) through which atmospheric air gets into the device 100 during respiration. As best seen in FIG. 7, the suction body 102 includes a check valve 104, a first filter 106, and a first casing 108 through which atmospheric air is forcibly introduced within a main body 110 of the device 100 during respiration process. The main body 110 of the device 100 includes a handle 110a debossed over the body 110. The handle 110a facilitates a user of the device 100 to comfortably grip the device 100. The filter 106 comprises a mesh filter or a filter made of nonwoven fabric or of like nature. The check valve or the back cover 104 is preferably made of but not limited rubber, and urethane having elasticity and restoring force. According to the embodiment, the check valve 104 is half-moon shaped and consists of an opening 104a at its centre. According to an embodiment, the opening 104a is preferably in a cross or plus shape. In operation, the opening 104a is opened and closed by the pressure of breathing or inhalation and due to the elasticity of the material of the check valve 104.

In an assembled configuration of the device 100 as seen in FIGS. 1-4 in conjunction with FIG. 7, the first filter 106 and the check valve 104 are engaged or mounted over the casing 108. Further, during assemblage, the casing 108 is operatively coupled to a distal end of the main body 110. The casing 108 is threaded (threads 108a) externally which rotatably engages to threading (not seen) present within the distal end of the main body 110. The casing 108 couples at the distal end of the main body 110 of the device 100 using a gasket creating a seal for the atmospheric air entering and passing through the device 100. The gasket is a loop of elastomer with a round cross-section that seated in a groove and compressed during assembly between the casing 108 and the main body 110. According to the embodiment, the gasket is an O-ring 125.

According to the embodiment, the hand held oxygen generating and air purifying device 100 further includes a solid oxygen filter 112 adapted for holding solid oxygen contained therein. The solid oxygen includes a compound preferably but not limited to calcium hydroxide, calcium oxide, potassium excess, calcium peroxide, magnesium peroxide, potassium peroxide, barium peroxide, and sodium peroxide. The solid oxygen compound obtained by redox reaction usually have small particle size of several micro (μm), which makes it difficult to breathe through the mouth and does not generate much oxygen upon reacting with atmospheric air. Thus, according to the best mode of operation, it is preferable to have the solid oxygen in a tablet of about 1˜5 mm particle size and this can be made using a granulator.

Further, according to the embodiment, the solid oxygen filter 112 is configured for holding the solid oxygen. The solid oxygen filter 112 is cylindrical in shape and is preferably made of nonwoven fabric or a mesh net.

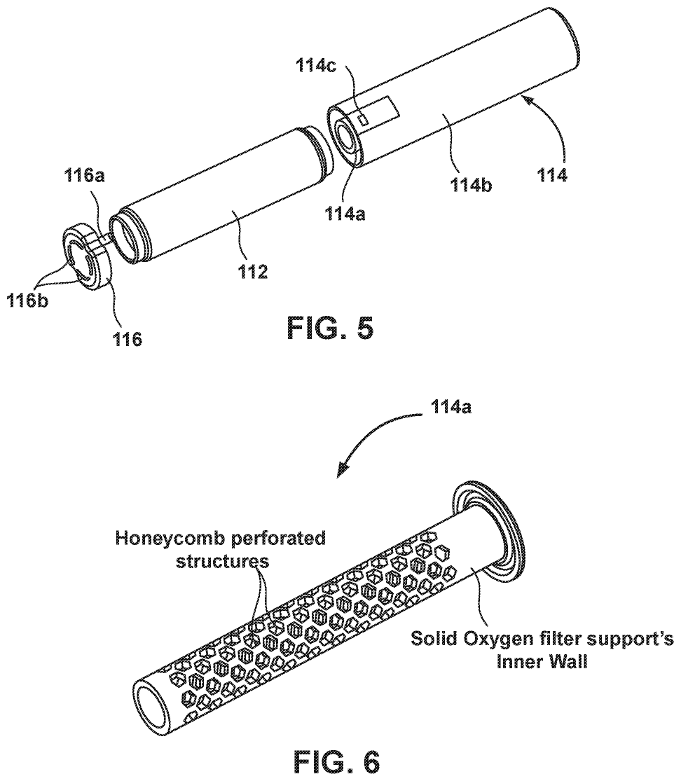

Further, according to the embodiment, the oxygen generator 100 includes a solid oxygen filter support 114 having a dual cylindrical structure configured for housing and supporting the solid oxygen filter 112. The dual cylindrical structure 114a, 114b of the solid oxygen filter support 114 includes an inner cylinder 114a having a honeycomb perforated structure over its wall, and an outer cylinder 114b having a blocked or solid outer wall surface as seen in FIGS. 4 and 5. The solid oxygen filter support 114 further includes a pair of slots (such as a slot 114c) for engaging to a pair of hooks 116a of a solid oxygen filter support lid 116.

The solid oxygen filter support lid 116 (seen in FIG. 5) is mountable over the solid oxygen filter support 114 and engages thereto using the pair of hooks 116a of the lid 116. Although only one hook is visible, but it should be understood there are two for holding the lid 116 over the filter support 114. The solid oxygen filter support lid 116 further includes a set of openings or slits 116b. In an example, the slits 116b may be configured in a circular configuration for discharging the generated inhalable oxygen from the main body 110 to a second filter or micro filter 118 during the operation of the device 100. The micro filter 118 is tubular in shape and consists of micro fibres. The micro filter 118 removes contaminants present in the generated oxygen and prevent entry of moisture from outside environment while the device 100 is being used for oxygen generation and inhalation of generated oxygen.

According to the embodiment, as seen in FIGS. 4 and 8 (represented as Section A), the oxygen generator device 100 further includes a second casing 120 embodying the second filter/micro filter 118 and is operationally connected to a mouthpiece 122. During the assembly as shown in FIG. 1, the casing 120 is operatively coupled to a proximal end of the main body 110 of the device 100. The casing 120 is threaded (threads 120a) externally which rotatably engages to threading (not seen) present within the proximal end of the main body 110. The casing 120 couples at the proximal end of the main body 110 of the device 100 using a gasket creating a seal for the atmospheric air entering and passing through the device 100. The gasket is a loop of elastomer with a round cross-section that seated in a groove and compressed during assembly between the casing 120 and the main body 110. According to the embodiment, the gasket is an O-ring 130.

Further, the mouthpiece 122 connected to the casing 120 (that embodies the micro filter 118) includes an extension 124. The extension 124 facilitates mounting of a straw 126 through which the user of device 100 can inhale the oxygen generated using the oxygen generator 100. The straw 126 is made of Effective Microorganisms (EM) ceramic of probiotics. The EM ceramic is composed of aluminium silicon oxide having an orthorhombic crystal structure.

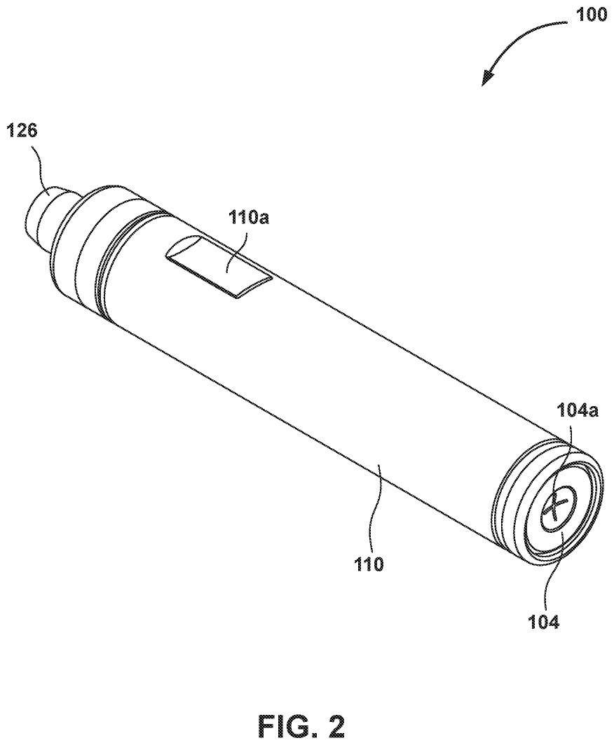

In operation, the hand held e-cigarette shaped oxygen generating and air purifying device 100 is used in an assembled form as seen in FIG. 1 or 2. The user will preferably hold the device 100 using the handle 110a and place the straw 126 in his mouth for breathing/inhalation of oxygen. During breathing or inhalation, the check valve 104 (in fact the opening 104a present in the check valve 104) will open up and allow the atmospheric air to forcibly enter inside the device 100. The atmospheric air then passes though the suction body 102, particularly the filter 106 and casing 108. The filter 106 will filter the contaminant present in the atmospheric air entering the device 100. The atmospheric air is then forced to pass through the internal cylinder 114a having honeycomb perforated structure and react with the solid oxygen content present inside the solid oxygen filter 112 to generate inhalable oxygen while the user is breathing. Technically, Oxygen is generated by reaction of solid oxygen compounds such as calcium hydroxide, calcium oxide, potassium peroxide, calcium peroxide, magnesium peroxide, potassium peroxide, barium peroxide and sodium peroxide.

Among these, excess potassium oxide reacts with moisture and carbon dioxide at room temperature to generate oxygen as shown in the reaction formula below:

2KO2+H2O→2KOH+3/2 02

2K02+CO2→K2C03+3/2 02

That is, carbon dioxide is removed and oxygen is generated at the same time. In addition, the oxygen generated as shown above may oxidize formaldehyde to form formic acid to remove formaldehyde seen in the reaction formula below:

HCHO+½O2→HCOOH

The formaldehyde removal reaction by potassium superoxide does not generate formaldehyde again in a variable manner, so it is more efficient for removing harmful gases than activated carbon and may be more useful depending on the purpose of use.

At this time when the oxygen is being generated, if required, one can add fragrance to colorless and odorless oxygen by adding into the solid oxygen filter 112. Some examples of the fragrances include but not limited to menthol and nicotine. Further, once the oxygen is generated by reacting the atmospheric air with the solid oxygen contained insider the solid oxygen filter 112, the generated oxygen is further passed through the micro filter 118 to further remove contaminants present in the generated oxygen and prevent entry of moisture from outside environment. This dual filter (the micro filter 118 and solid oxygen filter 112) configuration ensures delivery of purified oxygen to the user breathing using the device 100. The solid carbon filter 112 used in the proposed invention provides a large reaction surface area (for atmospheric air to react with solid oxygen).

According to some embodiment, the proposed oxygen generator 100 may be usable with a breathing exercising mobile application communicatively coupled with the device 100 and the device 100 can be used by users to develop sound breathing/air inhalation pattern often required during air inhalation exercises.

While various embodiments of the present invention have been illustrated and described, it will be clear that the present invention is not limited to these embodiments only. Numerous modifications, changes, variations, substitutions, and equivalents will be apparent to those skilled in the art, without departing from the spirit and scope of the present invention, as described in the claims.

What is claimed is:

1. A portable hand held e-cigarette shaped oxygen generating and air purifying device (100), comprising:
a suction body (102) having a check valve (104), a first filter (106), and a first casing (108) through which atmospheric air is forcibly introduced within a main body (110) of the device (100) during respiration, the first filter (106) and the check valve (104) are mounted over the first casing (108), and wherein the first casing (108) is operatively coupled to a distal end of the main body (110);
a solid oxygen filter (112) adapted for holding solid oxygen therein, and a solid oxygen filter support (114) having a dual cylindrical structure and configured for housing and supporting the solid oxygen filter (112) therein;
a solid oxygen filter support lid (116) with a pair of hooks (116a), the solid oxygen filter support lid (116) is mounted over the solid oxygen filter support (114), wherein the solid oxygen filter support lid (116) further configured to discharge generated inhalable oxygen from the main body (110) to a second filter (118);
a second casing (120) embodying the second filter (118) and operationally connected to a mouthpiece (122) and coupled to a proximal end of the main body (110), and wherein the mouthpiece (122) having an extension (124) for mounting a straw (126) thereon; and
wherein the atmospheric air introduced by respiration reacts with the solid oxygen contained inside the solid oxygen filter (112) to generate inhalable oxygen, wherein the atmospheric air reacts with the solid oxygen after passing through the dual cylindrical structure (114a,114b) of the solid oxygen filter support (114).

2. The device (100) of claim 1, wherein the solid oxygen filter (112) is made of nonwoven fabric or a mesh net and is fixed onto the solid oxygen filter support (114) and is configured to slide within the solid oxygen filter support (114) when the device (100) is assembled for usage.

3. The device (100) of claim 2, wherein the solid oxygen filter (112) may comprise of fragrances added therein.

4. The device (100) of claim 3, wherein the fragrances comprises of at least menthol and nicotine.

5. The device (100) of claim 1, wherein the first filter (106) is at least a mesh filter or a filter made of nonwoven fabric.

6. The device (100) of claim 1, wherein the first casing (108) and the second casing (120) are threaded for coupling to internal threading present within the distal end, and the proximal end of the main body (110) respectively.

7. The device (100) of claim 1, wherein the first casing (108) and the second casing (120) are coupled at the distal end and the proximal end of the main body (110) using a gasket creating a seal for the atmospheric air entering and passing through the device (100).

8. The device (100) of claim 7, wherein the gasket is an O-ring.

9. The device (100) of claim 1, wherein the solid oxygen filter support lid (116) comprising a set of openings (116b) for discharging the generated inhalable oxygen from the main body (110) to the second filter (118) during the operation of the device (100).

10. The device (100) of claim 1, wherein the dual cylindrical structure (114a, 114b) of the solid oxygen filter support (114) comprises an inner cylinder (114a) having a honeycomb perforated structure over its wall, and an outer cylinder (114b) having a solid outer wall surface.

11. The device (100) of claim 1, wherein the check valve (104) comprising materials selected from a group consisting of rubber, and urethane having elasticity and restoring force.

12. The device (100) of claim 1, wherein the check valve (104) is half-moon shaped and consists of an opening (104a) at its centre, wherein the opening (104a) is opened and closed by the pressure of breathing or inhalation.

13. The device (100) of claim 1, wherein the solid oxygen comprising of a compound selected from a group consisting of calcium hydroxide, calcium oxide, potassium excess, calcium peroxide, magnesium peroxide, potassium peroxide, barium peroxide, and sodium peroxide.

14. The device (100) of claim 13, wherein the solid oxygen is in a table form having a size of about 1˜5 mm.

15. The device (100) of claim 1, wherein the straw (126) is made of Effective Microorganisms (EM) ceramic of probiotics.

16. The device (100) of claim 15, wherein the EM ceramic is composed of aluminium silicon oxide having an orthorhombic crystal structure.

17. The device (100) of claim 1 further comprising a handle (110a) formed over the main body (110) for facilitating a user of the device (100) to comfortably grip the device (100).

18. The device (100) of claim 1, wherein the solid oxygen filter support (114) further comprises of a pair of slots for engaging to the pair of hooks of the solid oxygen filter support lid (116).

19. The device (100) of claim 1, wherein the second filter (118) is a micro filter tubular in shape and consisting of micro fibres for removing contaminants present in the generated oxygen and preventing entry of moisture from outside environment.
